(19) United States
(12) Patent Application Publication
(10) Pub. No.: US 2002/0193861 A1
(43) Pub. Date: Dec. 19, 2002

(54) A.C.-powered, user-manipulate-able electrodes for stimulating living tissue

(76) Inventor: Steven Michael Harrison, Montgomery, AL (US)

Correspondence Address: Steven Harrison, 3908 Croydon Rd., Montgomery, AL, 36109, US

(21) Appl. No.: 09/867,955

(22) Filed: May 30, 2001

(51) Int. Cl. A61N 1/04

(52) U.S. Cl. 607/153

(57) ABSTRACT

Improved electrodes for transmission of electrical current through living tissue for various therapeutic, analgesic, muscle training, and recreational uses. These user manipulate-able electrodes permit the use of low voltage alternating current (AC) instead of direct current (DC). The electrodes are manufactured by overlaying a conductive material with nonconductive semi-porous material which, when wetted, becomes conductive and serves as a skin moistening vehicle. Electrical resistance is controlled by adding layers of semi-porous material as desired. A potentiometer is available to the user for major adjustments to the current flow. These electrodes are supplied power by means of less complex and cheaper Alternating Current power supplies. In addition to the handheld version, there are other configurations intended for various uses.

### CROSS REFERENCE TO RELATED APPLICATIONS

Not Applicable

### STATEMENT OF FEDERALLY SPONSORED RESEARCH/DEVELOPMENT

None

### REFERENCE TO MICROFICHE APPENDIX

None

### BACKGROUND

1. Field of Invention

This invention relates to electrodes, specifically to electrodes that are used for stimulation of living tissue for medical purposes, analgesia, research, recreation or muscle retraining and development.

2. Description of Prior Art

Numerous patents have been issued for various electrodes to stimulate living tissue for diverse purposes. These include treatment of incontinence, sexual arousal, pain relief, muscle toning and training, and numerous therapies such as relaxation and sleep conditioning.

The art is replete with various apparatus for the supply and delivery of controlled electrical current to numerous sites on and within the body of the user or patient. This includes complex electrical circuitry in the power supplies, and use of synthetic materials in electrodes (refer to PES electrode patent). In electro-stimulation, precise current control is normally achieved by means of sophisticated power supplies equipped with extensive controls for modifying the waveform and timing of the output current. Besides considerable expense, this presents the user with the need to adjust the current flow at the power supply end, which calls for proximity and expertise.

Direct current (DC) is the prevalent form of current used for electro stimulation. However, due to its mono-phasic nature, direct current requires modification for use in electro stimulation. It is more easily modified for this purpose than AC, but this entails an expensive and complicated power supply. Alternating current, because of its continuous phase reversal aspect is, in some ways, more suited for electro-stimulation. However, due to various aspects such as portability, safety issues, and limitations of prior art electrodes, it has historically been a second choice to D.C. Prior art electrodes are quite advanced, but they are designed to conduct the amount of current supplied, and to provide little or no resistance. Controls on the (D.C.) power supply must be used to make adjustments to current flow.

Most electrodes used in electro stimulation are a skin-adhesive patch or button that is taped, or adhered to the body, and are generally used in medical applications. This type requires use of a conductive gel to make the subject skin suitably conductive and to promote continuous contact between the electrode and the user's skin. Other electrodes require penetration of a body orifice. This type must generally be deployed in close proximity to an internal organ. Still others, of varying descriptions, are for external use. All of these electrodes have a fixed conductivity, of minimal resistance, that is not adjustable by the end user. They require an adjustable output power supply for this reason. Prior art electrodes, particularly the recreational variety, and adjustable D.C. power supplies, are expensive.

In medical applications it is essential to have precise control mechanisms, and consistent electrodes for application of the electrical current to the patient. In fact, complex circuitry and multiple controls on power supplies are the norm for electro stimulation appliances described in the prior art and currently available on the market. That is, most prior art electrodes rely on expensive and complicated power supplies. Also, the use of exotic materials and elaborate fabrication techniques for electrodes is another aspect of prior art that causes these electrodes to be expensive.

The above considerations present several problems for potential users of these devices.

First, the power supply units are expensive. Currently available electrodes require the use of TENS-type power supplies. These, when coupled with the cost of the electrodes, elevate the cost of a complete system to between $300 and $900.

Second, DC-power supplies, although providing a great deal of control, are not straightforward in actual use and thus require a knowledgeable assistant or professional, at least until the most beneficial settings are found.

Third, some electrodes require insertion into a body orifice. This presents several problems:

a) health risks if proper septic regimen is not scrupulously employed;

b) careful and exact placement of the electrode against an internal organ to achieve optimum performance results;

c) possible injury to an internal organ, or perforation, if the inserted electrode is mismanaged;

d) rectal pain;

e) user discomfort and distaste for invasive penetration; and

f) reluctance to experiment with recreational applications because of the invasive nature of this type of electrode;

g) great caution must be exercised in adjustment of the DC power supply to avoid painful levels of electrical current.

Fourth, electrodes that do not require insertion, usually require affixing to the subject skin with adhesives or tapes. This limits the area of application to the one contact area on the skin where the electrode is affixed. Also, a conductive gel must be used in order to ensure continued contact between the skin and electrode. Patch-type electrodes that rely on tapes and adhesives are awkward and uncomfortable during use and removal.

Fifth, neither insert-able electrodes nor adhesive electrodes give the user control of the electrodes such that they can be manipulated while in use to stimulate varying sites on the anatomy or to enhance effects through movement around a particular site on the body.

Sixth, for use in recreational stimulation or personal therapy, products currently offered on the market are not commonly provided as one simple complete bundled product. The potential user is confronted with a dizzying array of components, which must be combined for use in order to have a complete functional system.

The problems identified above are solved by my manipulate-able electrode by:

(a) providing an adjustable means of controlling electrical current flow at the point of delivery,

(b) providing a means to wet the subject skin as the electrode is moved about, such that the need for conductive gel, fixed placement and adhesives are obviated,

(c) providing an electrode shape that enables it to be handheld and manipulated,

(d) providing a complete system that is simple enough, economical enough, and safe enough for a novice to use effectively,

(e) providing for use of Alternating Current where prior art electrodes tend to require Direct Current.

The above objectives are accomplished by use of a layer-able, semi-porous, nonconductive material, which becomes conductive when wet. This material is used to cover a fully conductive electrode. In practice, it was found that very few semi-porous materials provide sufficient resistance to be of use in this application. Doubtless, one skilled in the art could utilize other materials, but my electrode incorporates 3M Micropore cloth adhesive tape. It is capable of becoming saturated and yet has good resistance characteristics. Second, this covering permits the use of Alternating Current which is not normally useable as a direct stimulation power source. Last, the shape of the electrodes are such that they can be handheld and manipulated about an anatomical site. These combined aspects achieve a unique electro stimulation effect unlike any other.

### SUMMARY

In accordance with the present invention, a pair of electrodes consisting of a conductive material overlaid with a nonconductive semi-porous material which, when wetted, becomes conductive, serves as a skin-moistening vehicle, and provides the ability to adjust resistance by adding more layers of semi-porous material. Further these electrodes may be handheld and manipulated; do not require insertion, or the use of fixatives or conductive gels, are powered by an inexpensive A.C. output, step-down transformer, and provide more natural, orgasm-like sensations for the user.

### OBJECTS AND ADVANTAGES

The present invention offers the user a handheld, manipulate-able electrode that can utilize the peculiar and beneficial aspects of Alternating Current as a power source. The electrodes are economical to produce, and can be modified by the user to consistently supply the desired amount of resistance to suit the individual user. The combination of an economical AC power supply and low cost electrodes will reduce the cost to the user by as much as 80% and is much simpler to use. Line-voltage shock hazards are eliminated by use of an isolated step-down transformer power supply. With the electrical plug incorporated into the power supply housing, this device is safe enough even for use in wet environment such as a bathroom.

The objects and advantages of the present invention are:

(a) use of applied material coverings on electrodes, which require a wetting solution to become conductive and which serve as moisture delivery vehicle to subject skin tissue;

(b) use of electrode coverings which, by their layered nature, permit adjustment of the amount of current received by the end user;

(c) electrodes which can be handheld by the user who, by varying the amount of pressure holding the electrodes in contact with the skin, and moving the electrodes about on the surface of the skin, can achieve excellent electro-stimulation results unlike those described in prior teachings;

(d) an economical product relative to previous ones;

(e) a simple and straightforward device, requiring no assistance or prior training;

(f) eliminates electrode-related health risks such as internal infection by obviation of need for body orifice insertion;

(g) simplifies use of the electrodes by obviating the need to properly place the electrode(s) against an internal organ;

(h) simplifies the use of the electrodes by eliminating the need to adhere a patch-type electrode to the skin;

(i) a more user-friendly electrode which, by design, lends itself to casual trial and experimentation by the novice user.

(j) a complete ready to use product which doesn't require extensive study and purchase of various separate components to have a functional system;

(k) an economical, easy-to-use product which produces orgasm-like sensations for the user.

(l) an AC-powered pair of electrodes which, among other therapeutic uses, can be used to exercise the pelvic floor muscle and thereby help overcome male urinary dysfunction and related problems.

(m) an AC powered pair of electrodes which can be used for analgesic purposes on various parts of the anatomy.

(n) an AC-powered pair of electrodes which can be used for research.

It is further an object of this patent to protect the electrode shape, i.e., the elongated hold-able shapes which make for ease of use on the human anatomy. Other electrodes of similar shape are intended for insertion, that is, internal use. The present invention is not. These electrodes are designed to be handheld to facilitate exploration and stimulation of the external anatomy by the user. They do not have the penile shape of a dildo or vibrator, but rather are intended to be comfortable to hold with the human hand while manipulating the electrically charged end on the anatomy. It is further stipulated that variations of electrode shape, including ones designed to strap on, and stay in a fixed position, so as to allow one to engage in sexual intercourse with a partner, are considered part of this claim and easily within the capability of those skilled in the art. The addition of the wettable, layer-able, semi-porous material to the electrodes, along with the hand-held, manipulate-able aspects, permit these electrodes to function differently than those hitherto known and described in prior teachings.

It is further stipulated that one skilled in the art could easily create an electrode capable of similar functioning characteristics by use of composite material for the conductive portion of the electrode which would limit conductivity to a desired value. This would not be user adjustable. Nor would it have a layer of material to serve as a moistening vehicle. Nor would it have a saturate-able layer. A further variation would include an electrode with little or no resistance combined with an adjustable variable resistor such as an audio potentiometer. Both variations would necessarily be more costly.

The above aspects, when taken together, make these electrodes different from all others. Still further objects and advantages will become apparent from consideration of the ensuing description and drawings.

### DRAWING FIGURES

Reference Numerals In Drawings

FIG. 1: (note: one electrode is shown, but two are required for use - the second one
does not include a potentiometer)

| | | | |
|---|---|---|---|
| 1 | nonconductive hollow tube | 4 | 3M Micropore cloth adhesive tape |
| 2 | power lead passing through tube | 5 | wire connector |
| 3 | exposed copper strands on exterior of tube | 6 | 10K Ohm potentiometer |

FIG. 2: (electrodes have same design as in FIG. 1/3)

| | | | |
|---|---|---|---|
| 1 | power leads | 4 | crotch harness |
| 2 | 10K Ohm potentiometer | 5 | scrotal electrode |
| 3 | wire connector | 6 | anal electrode |

FIG. 3:

| | | | |
|---|---|---|---|
| 1 | power lead | 4 | electrode |
| 2 | 10K Ohm potentiometer | 5 | securement belt |
| 3 | wire connector | 6 | securement buckle |

### DESCRIPTION

FIG. 1—Preferred Embodiment

The electrodes in the preferred embodiment consist of two separate tubes of nominal one by six inch dimensions with closed, round ends. They are equipped with small holes on either end. A length of wire passes through both ends. One rounded end of each tube has bare copper wire strands arrayed along its outer surface. The bare copper wire is covered with 3 m brand adhesive cloth-tape which is affixed to the body of the tube. The other end, with insulated wire protruding, is equipped with a wire connector, which can be attached to an AC output power supply. There is a 10K Ohm potentiometer installed in-line between the electrode and the wire connector. The preferred embodiment, as a marketed product, will include an AC output power supply, which offers two settings: 18 and 24 volts of 1,000 milliamps. Two voltage settings give the user even more control of stimulation without increasing cost dramatically. The improved electrodes can also be used with DC power supplies such as TENS units, but require adjustment as with prior art electrodes.

The preferred embodiment uses 3M Micropore Surgical Tape as the conductive covering because it has a uniform thickness and adhesive backing which does not inhibit conductivity excessively. It is a ready made conductive covering. Nonetheless, the concept extends to all porous materials one skilled in the art could substitute to achieve the same functionality. The chief factors are: use of a wetting agent and control of the amount of current which is allowed to pass to the anatomy by means of graduations of thickness, porosity, and moisture retention potential of the material employed. The feature of being hand-held is also important in that these electrodes are capable of being manipulated at will by the user to achieve a much broader range of sensation than if the electrodes were left in one stationary position. The ability to be manipulated is simply not practical with most other electrodes.

Advantages

From the description above, a number of advantages of my manipulate-able electrode become evident:

(a) The instant invention provides a means to stimulate living tissue for various health and recreational purposes using low voltage, fixed output AC current, thereby obviating the need for elaborate and expensive AC or DC power supplies which include complex circuitry for modifying and controlling power output.

(b) The instant invention can be used with more elaborate power supplies, but unlike prior art electrodes doesn't require a TENS-type power supply for proper functioning. The aspect of the invention which permits use of fixed output AC current is a semi-porous covering, over the conductive wiring, which becomes conductive when wet.

The simplicity and advantages of my manipulate-able electrodes will become readily apparent to those skilled in the art from the following detailed description of a preferred embodiment taken in conjunction with the drawings, in which:

FIG. 1 shows a single preferred embodiment of my manipulate-able electrode with a potentiometer and button snap connector on the power source end. Two identical electrodes are employed in actual use.

Additional Embodiments

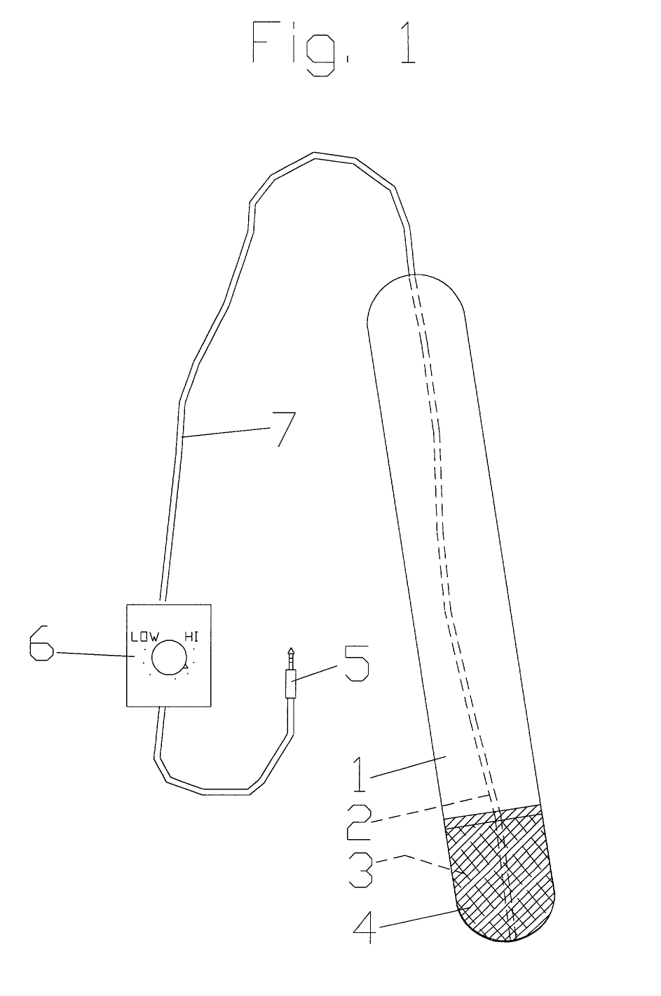

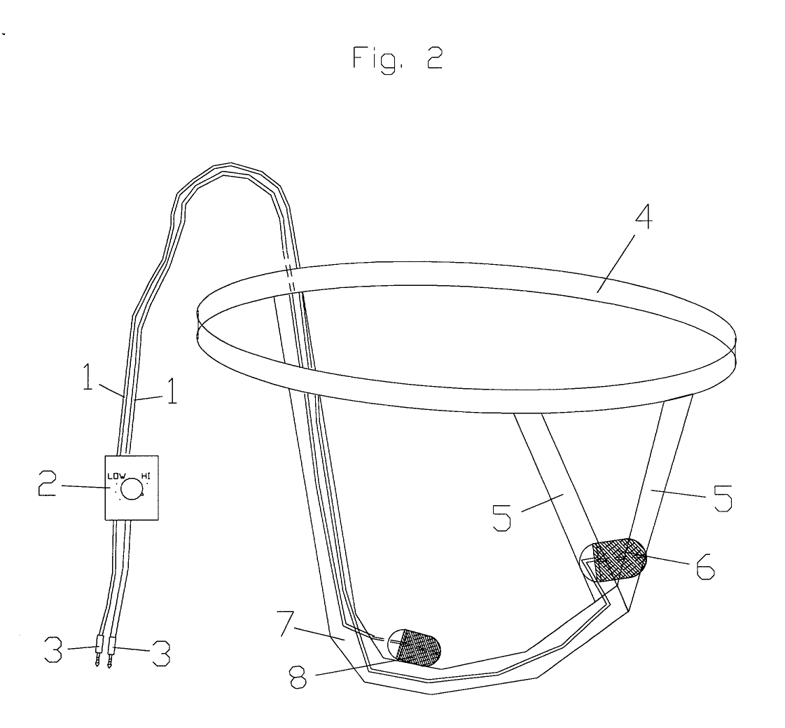

FIG. 2 shows an embodiment that can be worn while engaged in sexual intercourse.

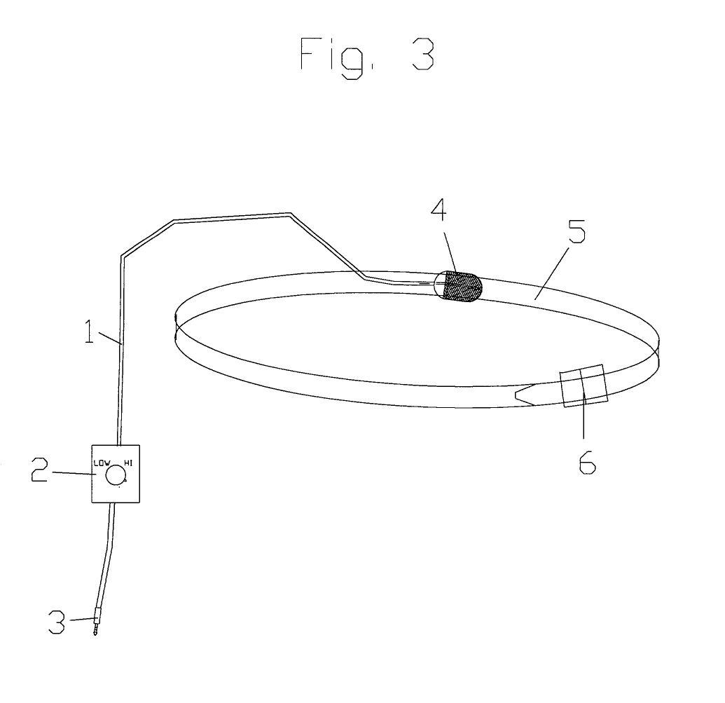

FIG. 3 shows an embodiment that can be attached to various parts of the body such as arms or legs.

Advantages

The crotch harness version allows the user to be highly stimulated for the duration of intercourse. The attachable version allows the user to deploy the electrode on the anatomy for therapeutic purposes without the need to hold it in place for the duration.

Operation

The tape-covered tips of the electrodes are briefly soaked in water to make them conductive. The tips, or sides, of the electrodes are then touched to the skin and pressure is applied to facilitate electrical conduction. On some areas of the anatomy, movement of the electrode about the site enhances stimulation. The electrodes can be placed on several locations of the anatomy to achieve intense stimulation. Among others, sensitive areas include the frontal hip pelvic region at the joint, the base of the scrotum, the female labia, the clitoral hood, the rear of the knee joints, the outer anus (but not inserted), and other areas below the waist. Most areas of the body will produce only minimal sensations when touched by the electrodes. Although use above the waistline is not currently recommended, no deleterious effects have been noted in prototype tests on subjects. The electrodes can be grasped by the user for movement around a particular site. This enhances the stimulation effect. Electrical hazard safety issues are eliminated by use of an isolated, step-down transformer that incorporates the plug in the power supply housing. If too much current is delivered, the user can add another layer of semi-porous tape on one or both of the electrodes to reduce current flow. Generally, two layers is ideal. Once the proper number of layers is established, this will be constant and there is no need for further adjustment. The tape is durable and will require refurbishment only infrequently.

Conclusion, Ramifications, and Scope

My manipulate-able electrodes described above, provide a safe, economical, less cumbersome, non-invasive, hand-held, user-friendly, means of stimulating various responses in the anatomy for purposes of therapy or recreation.

From the description above, numerous advantages of the improved electrodes are evident:

1. The electrodes are economical to produce relative to previous ones;

2. They excite in ways not obtained with direct current (DC);

3. They are used with cheaper and simpler AC isolated power supplies;

4. The entire system is simple and straightforward, requiring no assistance or prior training;

5. Electrode-related health risks such as internal infection are eliminated by obviation of need for bodily insertion;

6. Use of the electrodes is simplified by obviating the need to properly place the electrode(s) against an internal organ;

7. External use of the improved electrodes in the area of the anus, perineum, and scrotum produces results similar to insertion and direct contact with an internal organ such as the prostate gland.

8. Use of the electrodes is enhanced by eliminating the need to adhere a patch electrode to the skin;

9. These electrodes are handheld by the user and can be manipulated on various parts of the anatomy to achieve a broad range of stimulation;

10. These electrodes are user-friendly and, by design, lend themselves to casual trial and experimentation by the novice user.

11. The product is complete and ready to use and does not require extensive study and purchase of various separate components to have a functional system;

12. These electrodes produce natural orgasm-like sensations for the user.

While the above description contains many specificities, these should not be construed as limitations on the scope of the invention, but rather as an exemplification of one preferred embodiment thereof. Many other variations are possible. Accordingly, the scope of the invention should be determined not by the embodiment(s) illustrated, but by the appended claims and their legal equivalents.

What I claim is:

1. An improved electrode for providing electrical stimulation to living tissue comprising:
a nonconductive electrode body;
a conductive overlayment covering part of the exterior of the electrode body;
a nonconductive porous material, that, when wetted, becomes conductive and also serves as a skin moistening vehicle, covers the portion of the electrode body on which conductive material is present;
a 10K Ohm potentiometer is installed on one of the leads wires to one of the pair of electrodes for the purpose of fine-tuning current flow;
last, a single lead wire, equipped with connector, supplies current to the wire layer of the electrode.

2. Electrodes as per claim one in which the body of each electrode is made of a tubular length of nonconductive material with closed, rounded ends,
a conductive overlayment of stranded wire covers part of the exterior of the tubular electrode body;
a nonconductive porous material, 3M Micropore cloth adhesive tape, that, when wetted, becomes conductive and also serves as a skin moistening vehicle, covers the portion of the electrode tube which is overlaid with wire;
a single lead wire, equipped with connector, passes through the tubular body and supplies current to the wire overlayment and wetted porous material;

3. Electrodes as per claim two and additionally fitted with means of securement to the user's body by means of a harness which passes around the body and between the legs to temporarily secure the electrodes to the genital and crotch area thereby permitting use for the duration of intercourse with a partner;

4. Electrodes as per claim two and additionally fitted with means of securement of the electrodes to other locations on the body of the user such as on the legs, arms, neck, etc.
